(19) United States
(12) Patent Application Publication
(10) Pub. No.: US 2017/0290958 A1
(43) Pub. Date: Oct. 12, 2017

(54) PROCESS FOR FORMING A THERMALLY AND CHEMICALLY INERT MULTILAYER FILM

(71) Applicant: SWISS MEDICAL COATINGS SARL, Nidau (CH)

(72) Inventor: Edin Balic, Studen (CH)

(21) Appl. No.: 15/531,073

(22) PCT Filed: Nov. 25, 2015
(86) PCT No.: PCT/EP2015/077655

§ 371 Date: May 26, 2017

(30) Foreign Application Priority Data
Nov. 26, 2014 (CH) 08122/14

(51) Int. Cl. A61L 31/14; C08L 27/18; A61L 27/50; A61L 31/08; A61L 27/30; A61L 27/34

(52) U.S. Cl. CPC A61L 31/14; A61L 27/306; A61L 27/34; A61L 27/50; A61L 31/088; C08L 27/18

(57) ABSTRACT

A process for forming a thermally and chemically inert multi-layer film on a substrate, comprising depositing a composition comprising one or more inorganic oxide material, or mixtures thereof, on the substrate such as to form a continuous layer comprising a at least partially fused inorganic oxide material; depositing a composition comprising one or more non-fluorinated silane compounds, or mixtures thereof, on the continuous layer comprising a fused inorganic oxide material such as to form a layer comprising non-fluorinated polysiloxane; depositing a composition comprising one or more fluorinated silane compounds on the layer comprising non-fluorinated polysiloxane such as to form a layer comprising fluorinated silanes bearing a fluorinated group; depositing a composition comprising one or more copolymers of tetrafluoroethylene on the layer comprising fluorinated silanes bearing a fluorinated group such as to form a layer comprising a copolymer of tetrafluoroethylene.

### TECHNICAL FIELD

The present invention relates to a process for forming a thermally and chemically inert multi-layer film on a substrate, as well as to a substrate comprising on at least part of its surface a thermally and chemically inert multi-layer film.

### PRIOR ART

It is well-known in the art that many materials can be coated with certain chemical substances or mixtures thereof in order to confer a certain property to the thus coated materials. For instance, it can be advantageous to coat the surface of a substrate prone to corrosion with fluoropolymers which are well-known for their superior chemical inertness. The fluoropolymer layer can act as a barrier to the corrosive substances that may damage the underlying material if such a substance comes in to direct contact with the underlying material.

However, the most chemically resistant fluoropolymer, PTFE, cannot be melt processed and must therefore be applied to a given substrate in fine particulate form, in combination with a binder agent that encapsulates and holds the particles in place during use. Another method involves applying slurry of PFTE particles and sintering the particles on the substrates to form a layer of contiguous PFTE on the substrate.

However, the binder method has the inconvenience of introducing a less chemically inert material into a coating of PTFE, which will inevitably lessen the overall chemical resistance of the coating, while high sintering temperatures heavily restrict the choice of substrates. In any case, because it is difficult to achieve transparent coatings of such fluoropolymers their use in protective coatings for TV or computer screens and glasses is restricted.

WO2005/061023 relates to implantable medical devices having a fluorinated coating, and methods of coating the same. The fluorinated coating is made of an amorphous fluoropolymer coating comprising tetrafluoroethylene (TEF) and 4,5-difluoro-2,2-bis(trifluoromethyl)-1,3-dioxole, or a perfluoroelastomer. In order to form the fluorinated coating, the fluoropolymer is dissolved in a fluorinated organic solvent and the solution is applied to the medical device. The fluorinated coating is then formed by evaporation of the fluorinated organic solvent. However, such fluorinated coatings suffer from poor adhesion to the substrate, which means that these coatings can locally detach from the substrate because of mechanical stress, thereby allowing corrosive substances to enter into contact with the corrodible substrate. On the other hand, such coatings cannot handle repeated heating/cooling cycles such as the ones encountered in medical devices which are cleaned with aggressive disinfecting substances and autoclaved at high temperature between surgical procedures. WO2005/061023 discloses that the adhesion of the substrate to the fluoropolymer coating can be increased if a) a metal substrate surface is cleaned with a cleaning agent such as isopropanol, b) a metal substrate is subjected to plasma etching, chemical etching, or c) if a metal substrate is treated with perfluorodecyltriethoxysilane. Even though adhesion is partially increased by these treatments, it is nonetheless desirable to further increase the adhesion of fluoropolymer-based coatings to their substrate.

There exists thus a need to provide for a fluoropolymer-based coating or film that has good optical transparency and which is resistant against corrosive substances such as strong acids, while further displaying increased adhesion to a substrate when compared to existing solutions and which is preferably unaffected in said properties by frequent heat cycling.

### SUMMARY OF THE INVENTION

The present invention provides for a process of forming a chemically inert multi-layer film on a substrate which displays increased adhesion to its substrate; thereby providing a film that protects the coated substrate against thermal, mechanical and chemical stress for extended periods of time.

Within the context of the present disclosure, the term “thermally inert” refers to the capability of a material to be brought to a temperature of more than 200° C., preferably 250° C. or even 300° C., without thermal degradation.

Within the context of the present disclosure, the term “chemically inert” refers to the capability of a material to not chemically react with a chemical substance in contact therewith.

Within the context of the present disclosure, the term “non-porous” refers to a quality of a material in that the material has no pores or cavities in its bulk, i.e. is a solid essentially void of any interstices in the bulk.

The present invention provides for a process for forming a thermally and chemically inert multi-layer film on a substrate, comprising the steps of depositing a priming composition comprising one or more inorganic oxide material, or mixtures thereof, on the substrate such as to form a porous or fully densenon-porous continuous layer comprising an at least partially fused, one or more inorganic oxide material; optionally depositing a composition comprising one or more non-fluorinated silane compounds, or mixtures thereof, on the porous or non-porous continuous layer comprising at least partially fused inorganic oxide material such as to form a layer comprising non-fluorinated polysiloxane; optionally depositing a composition comprising a one or more fluorinated silane compounds on the layer comprising non-fluorinated polysiloxane such as to form a layer comprising fluorinated silanes bearing a fluorinated group; depositing a coating composition comprising one or more copolymers of tetrafluoroethylene on either the continuous layer comprising an at least partially fused inorganic oxide material, the layer comprising non-fluorinated polysiloxane, or the layer comprising fluorinated silanes bearing a fluorinated group such as to form a layer comprising a copolymer of tetrafluoroethylene. In a specific embodiment, the present invention further provides for a process for forming a thermally and chemically inert multi-layer film on a substrate comprising the steps of depositing a priming composition comprising one or more inorganic oxide material, or mixtures thereof, on the substrate such as to form a porous or non-porous continuous layer comprising at least partially fused one or more inorganic oxide material; depositing a composition comprising one or more non-fluorinated silane compounds, or mixtures thereof, on the porous or non-porous continuous layer comprising at least partially fused one or more inorganic oxide material such as to form a layer comprising non-fluorinated polysiloxane; depositing a composition comprising a one or more fluorinated silane compounds on the layer comprising non-fluorinated polysiloxane such as to form a layer comprising fluorinated silanes bearing a fluorinated group; depositing a coating composition comprising one or more copolymers of tetrafluoroethylene on the layer comprising fluorinated silanes bearing a fluorinated group such as to form a layer comprising a copolymer of tetrafluoroethylene.

The process according to the invention comprises the step of depositing a composition comprising one or more inorganic oxide material, or mixtures thereof, on the substrate such as to form a porous or non-porous continuous layer comprising at least partially fused of said one or more inorganic oxide material or mixtures thereof.

The substrates on which the thermally and chemically inert multi-layer film may be formed can be chosen from metals or alloys thereof such as for example surgical steels, aluminium, glass such as for example borosilicate, ceramics such as white ware ceramics or technical ceramics, thermoplastic polymers such as for example polyamide, polycarbonate, polyetheretherketone, treated or untreated elemental silicon, resins such as acrylic resin or phenolic resin, thermoset polymers such as NBR rubber, woven or non-woven textiles such as for example flash-spun polyethylene products, leather. Preferably, the substrates on which the thermally and chemically inert multi-layer film may be formed are metals or alloys thereof or ceramics. The substrate may also be a medical device such as a scalpel, lancet, stent, implant, or bone fixation means.

The composition comprising one or more inorganic oxide material may be in the form of a dispersion of one or more particulate inorganic oxide material in a suitable solvent or may consist of one or more particulate inorganic oxide material, eventually inorganic oxide material in the gas phase. In the case of a dispersion the solvent may be chosen from organic solvents or water. The composition comprising one or more inorganic oxide material can be deposited through dipping, spraying or other liquid deposition techniques and allowing the solvent to evaporate.

The inorganic oxide comprised in the composition comprising one or more inorganic oxide material, may be chosen from metal oxides, transition metal oxides or silica. Preferably, the inorganic oxide material is chosen from silica, alumina, titania, ceria, zirconia and mixtures of two or more. More preferably, the inorganic oxide material is chosen from fumed or precipitated silica or from alumina.

The continuous layer of at least partially fused inorganic oxide material may be formed by by conventional sintering, laser sintering or plasma spraying the composition comprising one or more inorganic oxide material.

In the case where the composition comprising one or more inorganic oxide material is formed into the continuous layer of at least partially fused of said one or more inorganic oxide material by applying the composition comprising one or more inorganic oxide material and subsequently conventionally sintering said composition comprising one or more inorganic oxide material, the composition comprising one or more inorganic oxide material is preferably in the form of a dispersion of a particulate inorganic oxide material in a suitable solvent. The dispersion may be applied through dipping, spraying or other suitable techniques and the dispersion may further be adjusted to a predetermined viscosity by the addition of thickening agents to achieve a desired coating thickness. The process of sintering per se is known, but for the general understanding the sintering process involves the heating of a particulate material to a temperature that is generally inferior to the melting temperature in order to enable molecular or atomic diffusion across the boundaries of the particles such that the individual particles fuse into a porous or fully dense continuous structure.

In the case where the composition comprising one or more inorganic oxide material is formed into the porous or non-porous continuous layer of at least partially fused inorganic oxide material by laser sintering, the composition comprising one or more inorganic oxide material preferably in the form of a dispersion of a particulate inorganic oxide material in a suitable solvent is deposited atop a suitable substrate through dipping, spraying or other liquid deposition techniques and allowing the solvent to evaporate. When the composition comprising one or more inorganic oxide material is laser sintered, power and focal point of the laser may be adjusted to regulate the temperature and/or size of the melt pool, depending on the inorganic oxide material used. The laser sintering process is known and understood to a person skilled in the art but for the general understanding the laser sintering process involves the heating of a particulate material to a temperature that is generally inferior to the melting temperature in order to enable molecular or atomic diffusion across the boundaries of the particles such that the individual particles fuse into a porous or fully dense continuous structure, and where the energy for this is supplied by a laser.

In the case where the composition comprising one or more inorganic oxide material is formed into the continuous layer of fused inorganic oxide material by applying the composition comprising one or more inorganic oxide material by plasma spraying, the substrate may be placed into a spraying chamber where the inorganic oxide material is partially molten and accelerated towards the substrate where it may form a porous or non-porous continuous layer of at least partially fused inorganic oxide material.

The term “at least partially fused inorganic oxide material” refers to an inorganic material that is formed by at least partially fusing a particulate inorganic oxide material into one continuous, solid body using preferably the above-mentioned methods. This may be achieved either by molecular or atomic diffusion across the boundaries of the particles or by partially melting the particles into semi-droplets or by providing enough energy to activate surface and bulk molecules to migrate towards the joining points between different touching inorganic oxide material constituents.

The process according to the invention may further optionally comprise the step of depositing a composition comprising one or more non-fluorinated silane compounds, or mixtures thereof, on the porous or non-porous continuous layer comprising at least partially fused inorganic oxide material such as to form a layer comprising non-fluorinated polysiloxane.

The non-fluorinated silane compounds of the composition comprising one or more non-fluorinated silane compounds may be chosen from halogenated silanes with the exception of fluorinated silanes, such as for example chlorosilanes, bromosilanes or iodosilanes, alkoxysilanes such as tetraalkoxysilanes like TEOS, aminosilanes such as APTES, APDEMS, APDMES, APTMS, glycidoxysilanes such as GPMES, mercaptosilanes such as MPTMS, MPDMS and silane (SiH₄).

In the case where one silane compound is present in the composition comprising one or more non-fluorinated silane compounds the silane compound is tetraethylorthosilicate (TEOS).

In the case where more than one silane compound is present in the composition comprising one or more non-fluorinated silane compounds, the silane compounds may be chosen to be silane (SiH₄) and dichlorosilane.

The composition comprising one or more non-fluorinated silane compounds may be in the form of a solution of one or more silanes in any suitable organic solvent. Preferably the organic solvent is an alkane or alkanol, more preferably ethanol or isopropanol.

The layer comprising a non-fluorinated polysiloxane may be formed by depositing the composition comprising one or more non-fluorinated silane compounds by a suitable method on the continuous layer comprising at least partially fused inorganic oxide material.

For instance, in the case where the composition comprising one or more non-fluorinated silane compounds is in the form of a solution, the composition comprising one or more non-fluorinated silane compounds may be applied through dipping, spraying or other liquid deposition techniques and allowing the solvent to evaporate.

For instance, in the case where the composition comprising one or more non-fluorinated silane compounds is in the form of a pure silane compound or a mixture of pure silane compounds, the composition comprising one or more non-fluorinated silane compounds may be applied through chemical vapour deposition. A person skilled in the art will know how the method of chemical vapour deposition can be adjusted to a given silane in order to obtain deposition thereof onto a substrate.

The process according to the invention may further optionally comprise the step of depositing a composition comprising one or more fluorinated silane compounds on the layer comprising non-fluorinated polysiloxane such as to form a layer comprising fluorinated silanes bearing a fluorinated group.

The fluorinated silane compounds of the composition comprising one or more fluorinated silane compounds may preferably be chosen from silanes bearing at least one perfluoroalkyl group, so-called perfluoroalkylsilanes. Preferably the fluorinated silane compounds are chosen from perfluoroalkylsilanes bearing at least one perfluoroalkyl group, at least one non-hydrolyzable group such as an alkyl or aryl group and at least one reactive group such as an alkoxy group, a halogen atom, hydroxyl group, amine or substituted amine group. More preferably the fluorinated silane compounds are chosen from perfluoroalkylsilanes bearing one perfluoroalkyl group, two linear or branched alkyl groups and one reactive group such as an alkoxy group, halogen, hydroxyl, amine or substituted amine group such as a dialkylamine group.

Preferably the perfluoroalkyl group of the perfluoroalkylsilane is a 1H, 1H, 2H, 2H-perfluoroalkyl group, more preferably a 1H, 1H, 2H, 2H-perfluoroalkyl group having a linear or branched alkyl chain of 3 to 10 carbon atoms.

In the context of the present disclosure, the term “1H, 1H, 2H, 2H-perfluoroalkyl group” refers to a alkyl group where every hydrogen atom has been replaced by a fluorine atom except the proximal hydrogen atoms at position 1, 1, 2, and 2 of the alkyl group.

Specific examples of suitable fluorinated silanes are 1H, 1H, 2H, 2H-perfluorodecyltrichlorosilane (CAS#78560-44-8) or 1H, 1H, 2H, 2H-perfluorooctyltriethoxysilane (CAS#51851-37-7) or more generally 111, 1H, 2H, 2H-perfluoroalkyldialkylhalogenosilanes or 1H, 1H, 2H, 2H-perfluoroalkyldialkylaminosilanes.

The composition comprising one or more fluorinated silane compounds may be in the form of a solution of one or more fluorinated silane in any suitable organic solvent. Preferably the organic solvent is an alkane or alkanol, more preferably ethanol or isopropanol.

The layer comprising fluorinated silanes bearing a fluorinated group may be formed by depositing the composition comprising one or more fluorinated silane compounds by a suitable method.

For instance, in the case where the composition comprising one or more fluorinated silane compounds is in the fouls of a solution, the composition comprising one or more fluorinated silane compounds may be applied through dipping, spraying or other liquid deposition techniques and allowing the solvent to evaporate.

For instance, in the case where the composition comprising one or more fluorinated silane compounds is in the form of a pure fluorinated silane compound or a mixture of pure fluorinated silane compounds, the composition comprising one or more fluorinated silane compounds may be applied through chemical vapour deposition. A person skilled in the art of will know how the method of chemical vapour deposition can be adjusted to a given fluorinated silane in order to obtain deposition thereof onto a substrate.

The deposition of the composition comprising one or more non-fluorinated silane compounds on the continuous layer of at least partially fused inorganic oxide material will yield a high areal density of reactive groups such as hydroxyl or alkoxy groups that are available to react with other compounds such as the one or more fluorinated silane compounds. Thus the optional deposition of both silane compounds and fluorinated silane compounds will create a fluorocarbon rich “molecular mat” on the substrate that will even further increase the adhesion of the copolymer of tetrafluoroethylene atop the created material sandwich, thereby ameliorating the thermal and chemical inertia of the fluoropolymer layer on the substrate.

The process according to the invention comprises the step of depositing a composition comprising one or more copolymers of tetrafluoroethylene either on the layer comprising fluorinated silanes bearing a fluorinated group or on the continuous layer of at least partially fused inorganic oxide material, depending on whether the thermally and chemically inert multi-layer film comprises merely a continuous layer of at least partially fused inorganic oxide material and a layer comprising a copolymer of tetrafluoroethylene or whether the thermally and chemically inert multi-layer film comprises a continuous layer of at least partially fused inorganic oxide material, a layer comprising non-fluorinated polysiloxane, a layer comprising fluorinated polysiloxane and a layer comprising a copolymer of tetrafluoroethylene.

The composition comprising one or more copolymers of tetrafluoroethylene may be in the form of a solution of one or more copolymers of tetrafluoroethylene in a suitable organic solvent, such as for example a perfluorinated solvent capable of dissolving the copolymers of tetrafluoroethylene. Examples of such solvents are perfluorinated alkanes or cycloalkanes, perfluorinated heterocyclic compounds, or mixtures thereof.

Thus the composition comprising one or more copolymers of tetrafluoroethylene preferably comprises a perfluorinated solvent dissolving the copolymers of tetrafluoroethylene, such as for example perfluorinated alkanes or cycloalkanes, perfluorinated heterocyclic compounds, or mixtures thereof.

The layer comprising one or more copolymers of tetrafluoroethylene may be formed by depositing the composition comprising one or more copolymers of tetrafluoroethylene by a suitable method.

The composition comprising one or more copolymers of tetrafluoroethylene may be in the form of a solution of one or more fluorinated silane in a perfluorinated solvent capable of dissolving the copolymers of tetrafluoroethylene.

For instance, in the case where the composition comprising one or more copolymers of tetrafluoroethylene is in the form of a solution, the composition comprising one or more copolymers of tetrafluoroethylene may be applied through dipping, spraying or other liquid deposition techniques and allowing the solvent to evaporate.

The copolymers of tetrafluoroethylene may be chosen from copolymers of tetrafluoroethylene and perfluoro-2,2-dimethyl-1,3-dioxole.

The copolymers of tetrafluoroethylene may be preferably chosen from copolymers of tetrafluoroethylene having a melting point in excess of 200° C., preferably of from 200 to 375° C., more preferably of from 240° C. to 360° and most preferably of from 320 to 360° C.

After the deposition of the compositions making up the layers of the multi-layer film, the thus formed multi-layer film may be heated to a temperature of 200° C. for 30 minutes, or 150° C. for 60 minutes to further increase adhesion.

The present invention further provides for a substrate having on at least part of it a thermally and chemically inert multi-layer film comprising, in this order, a continuous layer comprising at least partially fused inorganic oxide material; and a layer comprising a copolymer of tetrafluoroethylene; wherein the continuous layer comprising a at least partially fused inorganic oxide material is in contact with the substrate, and layer comprising a copolymer of tetrafluoroethylene is in contact with the continuous layer comprising a at least partially fused inorganic oxide material.

The present invention further provides for a substrate having on at least part of it a thermally and chemically inert multi-layer film comprising, in this order, a continuous layer comprising a at least partially fused inorganic oxide material; optionally a layer comprising non-fluorinated polysiloxane; optionally a layer comprising fluorinated polysiloxane; a layer comprising a copolymer of tetrafluoroethylene; wherein the continuous layer comprising a at least partially fused inorganic oxide material is in contact with the substrate, and the layer comprising a copolymer of tetrafluoroethylene is either in contact with the layer comprising the non-fluorinated polysiloxane, the layer comprising fluorinated silanes bearing a fluorinated group or the porous or non-porous continuous layer comprising at least partially fused one or more inorganic oxide material.

The present invention further provides for a substrate having on at least part of it a thermally and chemically inert multi-layer film comprising, in this order, a continuous layer comprising a at least partially fused inorganic oxide material ; a layer comprising non-fluorinated polysiloxane; a layer comprising fluorinated polysiloxane; a layer comprising a copolymer of tetrafluoroethylene; wherein the continuous layer comprising a at least partially fused inorganic oxide material is in contact with the substrate, and the layer comprising non-fluorinated polysiloxane is in contact with the continuous layer comprising a at least partially fused inorganic oxide material, and the layer comprising fluorinated polysiloxane is in contact with the layer comprising non-fluorinated polysiloxane, and the layer comprising a copolymer of tetrafluoroethylene is in contact with the layer comprising fluorinated polysiloxane.

The substrate is a glass, ceramic, polymer or metal substrate, preferably a metal medical device.

1. A process for forming a thermally and chemically inert multi-layer film on a substrate, comprising the steps of
a. depositing a priming composition comprising one or more inorganic oxide material, or mixtures thereof, on the substrate such as to form a porous or non-porous continuous layer comprising an at least partially fused, one or more inorganic oxide material;
b. optionally depositing a composition comprising one or more non-fluorinated silane compounds, or mixtures thereof, on the porous or non-porous continuous layer comprising at least partially fused one or more inorganic oxide material such as to form a layer comprising non-fluorinated polysiloxane;
c. optionally depositing a composition comprising a one or more fluorinated silane compounds on the layer comprising non-fluorinated polysiloxane such as to form a layer comprising fluorinated silanes bearing a fluorinated group;
d. depositing a coating composition comprising one or more copolymers of tetrafluoroethylene on either the continuous layer comprising an at least partially fused inorganic oxide material, the layer comprising non-fluorinated polysiloxane, or the layer comprising fluorinated silanes bearing a fluorinated group such as to form a layer comprising a copolymer of tetrafluoroethylene.

2. The process for forming a thermally and chemically inert multi-layer film on a substrate according to claim 1, comprising the steps of
a. depositing a priming composition comprising one or more inorganic oxide material, or mixtures thereof, on the substrate such as to form a porous or non-porous continuous layer comprising at least partially fused one or more inorganic oxide material;
b. depositing a composition comprising one or more non-fluorinated silane compounds, or mixtures thereof, on the porous or non-porous continuous layer comprising at least partially fused one or more inorganic oxide material such as to form a layer comprising non-fluorinated polysiloxane;
c. depositing a composition comprising a one or more fluorinated silane compounds on the layer comprising non-fluorinated polysiloxane such as to form a layer comprising fluorinated silanes bearing a fluorinated group;
d. depositing a coating composition comprising one or more copolymers of tetrafluoroethylene on the layer comprising fluorinated silanes bearing a fluorinated group such as to form a layer comprising a copolymer of tetrafluoroethylene.

3. The process according to claim 1 or 2, wherein the inorganic oxide is chosen from metal oxides, transition metal oxides or silica, preferably from silica, alumina, titania, ceria, zirconia and mixtures thereof.

4. The process according to claim 1 or 2, wherein the porous or non-porous continuous layer of at least partially fused inorganic oxide material is formed by sintering, laser sintering, or plasma spraying the composition comprising one or more inorganic oxide material.

5. The process according to any preceding claim, wherein non-fluorinated silane compound is chosen from chloro- bromo- or iodo-alkoxysilanes, aminosilanes such as APTES, APDEMS, APDMES, APTMS, glycidoxysilanes such as GPMES, and mercaptosilanes such as MPTMS, MPDMS and alkoxysilanes; and preferably is a tetraalkoxysilane such as TEOS.

6. The process according to any preceding claim, wherein the fluorinated silane compound is chosen from perfluoroalkylsilanes bearing at least one perfluoroalkyl group, at least one non-hydrolyzable group and at least one reactive group, preferably from perfluoroalkylsilanes bearing at least one perfluoroalkyl group, at least one alkyl or aryl group and at least one alkoxy group, a halogen atom, hydroxyl group, amine or substituted amine group.

7. The process according to any preceding claim, wherein the composition comprising one or more copolymers of tetrafluoroethylene further comprises a perfluorinated solvent dissolving the copolymers of tetrafluoroethylene, such as for example perfluorinated alkanes or cycloalkanes, perfluorinated heterocyclic compounds, or mixtures thereof

8. The process according to any preceding claim, wherein the copolymer of tetrafluoroethylene is chosen from copolymers of tetrafluoroethylene and perfluoro-2,2-dimethyl-1,3-dioxole.

9. A substrate comprising on at least part of its surface a thermally and chemically inert multi-layer film comprising in this order
a. a continuous layer comprising at least partially fused inorganic oxide material;
b. a layer comprising a copolymer of tetrafluoroethylene;
wherein the continuous layer comprising an at least partially fused inorganic oxide material is in contact with the substrate, and the layer comprising a copolymer of tetrafluoroethylene is in contact with the porous or non-porous continuous layer comprising at least partially fused one or more inorganic oxide material.

10. A substrate comprising on at least part of its surface a thermally and chemically inert multi-layer film comprising in this order
a. a continuous layer comprising at least partially fused inorganic oxide material;
b. an layer comprising non-fluorinated polysiloxane;
c. an layer comprisingfluorinated silanes bearing a fluorinated group;
d. a layer comprising a copolymer of tetrafluoroethylene;
wherein the continuous layer comprising at least partially fused inorganic oxide material is in contact with the substrate, and the layer comprising non-fluorinated polysiloxane is in contact with the porous or non-porous continuous layer comprising a fused inorganic oxide material, and the layer comprising fluorinated silanes bearing a fluorinated group is in contact with the layer comprising non-fluorinated polysiloxane, and the layer comprising a copolymer of tetrafluoroethylene is in contact with the layer comprising fluorinated silanes bearing a fluorinated group.

11. The substrate according to claim 9 or 10, wherein the at least partially fused inorganic oxide material of the porous or non-porous continuous layer is chosen from alumina, titania, ceria, silica, or mixtures thereof, and in particular is alumina or silica formed by sintering, laser sintering, or plasma spraying alumina or silica on the substrate.

12. The substrate according to claim 10, wherein the non-fluorinated silane for obtaining the layer comprising non-fluorinated polysiloxane is chosen from tertraalkoxysilanes, or mixtures thereof, and in particular from TEOS.

13. The substrate according to claims 10 to 12, wherein the copolymer of tetrafluoroethylene is a copolymer of tetrafluoroethylene and perfluoro-2,2-dimethyl-1,3-dioxole.

14. The substrate according to claims 10 to 13, wherein the substrate is a glass, ceramic or metal substrate, preferably a metal medical device.
